(19) United States
(12) Patent Application Publication
(10) Pub. No.: US 2007/0299383 A1
(43) Pub. Date: Dec. 27, 2007

(54) Foam layer cohesive articles and wound care bandages and methods of making and using same

(75) Inventors: Thomas S. Murphy, Boxford, MA (US); Michael Miller, York, ME (US)

Correspondence Address: WILMERHALE/BOSTON, 60 STATE STREET, BOSTON, MA, 02109, US

(73) Assignee: Andover Coated Products, Inc., Salisbury, MA (US)

(21) Appl. No.: 11/809,469

(22) Filed: Jun. 1, 2007

Related U.S. Application Data
(60) Provisional application No. 60/809,925, filed on Jun. 1, 2006.

(51) Int. Cl. A61F 13/02

(52) U.S. Cl. 602/46

(57) ABSTRACT

A foam pad attached to a portion of an elastically expansible cohesive article, e.g., a tape or bandage, the pad is placed over a wound, sore, or ulcer, and the remainder of the cohesive tape or bandage is wrapped around the afflicted body part, and the tape or bandage compresses the pad against the wound. Preferably, the foam pad is hydrophilic foam, the oppositely facing surfaces of the tape or bandage to which the pad is attached are each coated with a cohesive composition, the pad has a thickness greater than about 0.1 inches and is sized and adapted to cover a wound on a body part, and the longer elastically expansible tape or bandage is sized to wrap several times around the body part. The product may be provided as a roll in which the cohesive coated surfaces contact and cohesively adhere to each other, and may be in sterile condition in a sealed package. If intended for a single use, the product may include only a single pad.

### CROSS-REFERENCE TO RELATED APPLICATIONS

This application claims priority under 35 U.S.C. §119(e) to U.S. Provisional Patent Application No. 60/809,925, filed on Jun. 1, 2006 and entitled “Cohesive Articles with a Foam Layer,” the entire contents of which are incorporated herein by reference.

This application is related to U.S. patent application Ser. No. (TBA) entitled “Foam Layer Cohesive Articles and Wound Care Bandages And Methods of Making and Using Same,” filed concurrently herewith, and to U.S. patent application Ser. No. (TBA) entitled “Foam Layer Cohesive Articles and Wound Care Bandages And Methods of Making and Using Same,” filed concurrently herewith, the entire contents of which are incorporated herein by reference.

### BACKGROUND

1. Field

This application generally relates to cohesive articles, such as medical bandages.

2. Related Art

Tapes and bandages are frequently used in medical and sports applications requiring a strong and reliable, yet comfortable and easily applied, means of securing a limb or other body segment for prolonged periods of time. For example, strains and sprains can cause inflammation and the accompanying accumulation of fluid around a sprained joint. Wrapping the affected joint securely with an elastic bandage can prevent excess fluid from accumulating and causing additional tissue damage. In addition, leg ulcers of various origins, including venous stasis ulcers, arterial (ischemic) ulcers and neurotrophic (diabetic) ulcers, are a common medical problem. These leg ulcers are wounds or open sores that do not heal, or otherwise recur repeatedly, and cause persistent swelling as well as burning, itching, irritation and discoloration of the skin. Therapy for leg ulcers generally includes topical protection of the wound, as well as compression and antimicrobial treatment of the affected area.

There are a number of tapes and bandages that are available for such applications. For example, CO-FLEX (registered trademark of Andover Healthcare, Salisbury, Mass.) bandages and tapes are typically used in sports medicine, first-aid, and veterinary applications. In order to facilitate application, the tape or bandage should be relatively easy to tear to any length by hand across the width, but at the same time must have sufficient longitudinal strength. CO-FLEX tapes and bandages (see U.S. Pat. No. 3,575,782, the entire contents of which are incorporated herein by reference) have a laminated structure including two outer nonwoven layers with longitudinally-extending elastic yarns sandwiched between. POWERFLEX (registered trademark of Andover Healthcare, Salisbury, Mass.) bandages and tapes (see U.S. Pat. No. 5,762,623, the entire contents of which are incorporated herein by reference) include a layer that is elastic in the longitudinally-extending direction laminated to one side of a warp-knitted, sometimes referred to as warp-knitted (weft insertion), fabric oriented with the knit yarns extending longitudinally. Typically, the elastic layer of POWERFLEX tapes and bandages have longitudinally-extending elastic strands laminated between a pair of outer layers, at least one of which is warp-knitted (weft insertion). The warp-knitted (weft insertion) fabric has a weight of less than about 50 grams per square meter (about 1.5 oz. per square yard) and generally less than about 25 to 30 grams per square meter (about 0.7 to 0.9 oz. per square yard). The other outer layer of the tape or bandage is a lightweight spun-blown synthetic nonwoven. The complete tape/bandage has a good appearance, hand-tears cleanly transversely of the tape, has a longitudinal strength that on a weight basis is greater than that of commercially available hand-tearable woven bandage products, and is considerably less expensive than its woven or knitted competitors where the elastic yarns are woven or knitted into the fabric, as an integral part of the woven or knitted cloth.

Leg ulcers of various origins, including venous stasis ulcers, arterial (ischemic) ulcers and neurotrophic (diabetic) ulcers, are a common medical problem. These leg ulcers are wounds or open sores that do not heal, or otherwise recur repeatedly, and cause persistent swelling as well as burning, itching, irritation and discoloration of the skin. Therapy for leg ulcers generally includes topical protection of the wound, as well as compression and antimicrobial treatment of the affected area. Often, an absorbent pad is applied directly to an open wound, skin ulcer, or sore on a particular body part, taped in place, and used to absorb fluids emitted from such wound, ulcer, or sore.

### SUMMARY

The invention provides foam layer cohesive articles and wound care bandages and methods of making and using same.

Under one aspect, an elastically extensible cohesive article having first and second oppositely-facing exterior surfaces includes a foam layer; and a cohesive composition coating at least a portion of each of the first and second exterior surfaces of the article.

In some embodiments, the article includes a second layer juxtaposed with and secured with respect to at least a portion of the foam layer. The second layer may have first and second oppositely-facing surfaces, the first surface of the second layer being secured to the foam layer and the cohesive composition coating at least a portion of the second surface of the second layer. The second layer may include multiple layers, for example, an elastic layer and a layer including at least one of a knit fabric, a warp-knit fabric, a warp-knit weft-insertion fabric, a woven fabric and a non-woven fabric. The second layer may include at least one of a second foam layer, an elastic layer, an elastic fabric, a woven fabric, a knit fabric, a warp knit fabric, a warp-knit weft-insertion fabric, and a non-woven fabric. The cohesive composition may permeate the foam layer and the second layer, and secure the second layer relative to the foam layer. The second layer may be porous, such that portions of a surface of the foam layer coated with cohesive composition are exposed through the second layer. The cohesive composition may be present on at least a portion of at least one surface of the foam layer, and optionally on at least a portion of at least one surface of the second layer. A removable, non-cohesive release layer can be placed adjacent to at least one of the first and second surfaces of the article, for example if the article is wound into a roll.

In some embodiments, the foam layer has a thickness in the range of about 0.01 inches to about 0.25 inches, e.g., in the range of about 0.01 inches to about 0.01 inches, e.g., a thickness in the range of about 0.025 inches to about 0.035 inches. The foam layer may include a plurality of open cells that define at least a portion of at least one of the exterior surfaces of the article, the open cells having outwardly facing surfaces coated by the coating composition. The foam layer may define a portion of one of the exterior surfaces of the article. The article may have a length that is at least three times its width. The article may be wound into a roll such that the first surface is cohesively attached to the second surface. The cohesive composition may permeate the thickness of the foam layer. The cohesive composition may include at least one of a latex-based cohesive and a latex-free cohesive, e.g., at least one of natural rubber latex, synthetic rubber latex, polyisoprene, polychloroprene, polyester polyurethane, and polycaprolactone polyurethane. One of the first and second surfaces may bond to the other of the first and second surfaces with a peel strength of at least about 5 oz/in-w in a standard peel force test, e.g., between about 10 oz/in-w and about 40 oz/in-w, or between about 0.5 oz/in-w to about 14 oz/in-w.

The article may also include an absorbent pad juxtaposed with and secured to one of the first and second exterior surfaces of the article, and covering less than one-third of the one surface. The absorbent pad may include hydrophilic foam, which may include a plurality of open cells and may include at least one of polyurethane, silicone, polyethylene, and gauze. At least one of an adhesive, a cohesive, and a web adhesive can be used to secure the pad to the surface. A package may have a cohesive article sealed therein, and the article may be in a sterile condition.

Under another aspect, a method of making a cohesive article having oppositely facing first and second surfaces includes providing an elastically extensible foam layer having a thickness in the range of about 0.01 to about 0.25 inches; and applying a cohesive composition to the first and second surfaces of the article. The cohesive composition may be applied to the foam layer such that the cohesive composition permeates the thickness of the foam layer. At least one additional layer can be provided in juxtaposition with the foam layer and securing the additional layer and the foam layer to each other. The foam layer may include a plurality of open cells that define at least a portion of one of the exterior surfaces of the article and have outwardly-facing surfaces, and further comprising applying the cohesive composition to the foam layer such that the cohesive composition coats the outwardly-facing surfaces. The foam layer may have a thickness in the range of about 0.01 to about 0.1 inches, e.g., in the range of about 0.025 to about 0.035 inches.

Under another aspect, an elastically extensible cohesive article having first and second oppositely-facing exterior surfaces includes a foam layer having a thickness in the range of about 0.01 inches to about 0.1 inches and defining at least a portion of the first exterior surface of the article. The article also includes a second layer juxtaposed with and secured to the foam layer and defining at least a portion of the second exterior surface of the article, the second layer including at least one of a second foam layer, an elastic layer, an elastic fabric, a woven fabric, a knit fabric, a warp-knit weft insertion fabric, and a non-woven fabric. The article also includes a cohesive composition including at least one of a latex-based cohesive and a latex-free cohesive, the cohesive composition coating at least a portion of each of the first and second exterior surfaces of the article and permeating into the thickness of the article.

In some embodiments, the foam layer includes a plurality of open cells that define at least a portion of the first exterior surface, the open cells having generally outwardly facing surfaces and extending generally inwardly from the portion of the first exterior surface defined by the foam layer. The cohesive composition may coat the generally outwardly facing surfaces of a plurality of the open cells. The foam can be an open cell foam having a density, e.g., in the range of about 1 lb/ft³ to about 3 lb/ft³.

In some embodiments, the weight of the cohesive composition can be, e.g., in the range of about 20% to about 70% of the weight of the article, for example in the range of about 25% to about 45% of the weight of the article. The weight of the cohesive composition per square meter of a major surface of the article can be in the range of about 6 grams to about 70 grams, e.g., in the range of about 7.5 grams to about 36 grams. At least a portion of the foam may include a plurality of closed cells.

The length of the article may be not less than three times its width, and the article also may be wound into a roll with one of the surfaces of the article cohesively attached to the other of the surfaces of the article. The foam layer may have a thickness in the range of about 0.025 inches to about 0.035 inches.

The cohesive composition may include at least one of a latex-based cohesive and a latex-free cohesive, for example at least one of natural rubber latex, synthetic rubber latex, polyisoprene, polychloroprene, polyester polyurethane, and polycaprolactone polyurethane.

One of the first and second surfaces may bond to the other of the first and second surfaces with a peel strength of between about 10 oz/in-w and about 40 oz/in-w as measured in a standard peel force test, for example between about 0.5 oz/in-w to about 14 oz/in-w.

The article may have a weight in the range of about 30 to about 100 grams per square meter, e.g., in the range of about 40 to about 80 grams per square meter. An article may have a width of about 1 inch, about 1.5 inches, about 2 inches, about 3 inches, about 4 inches, or about 6 inches; and a length not less than about 36 inches.

A package may include the cohesive article sealed therein, and the article may be in a sterile condition. A removable, non-cohesive release layer may be placed adjacent to at least one of the first and second surfaces of the article.

The second layer may include multiple layers, for example an elastic layer and at least one of a knit fabric, a woven fabric, a warp-knit fabric, a warp-knit weft-insertion fabric, and a non-woven fabric. The article may also include a third layer juxtaposed with and secured to the foam layer and defining at least a portion of the first exterior surface of the article. The third layer may include at least one of a knit fabric, a warp-knit fabric, a warp-knit weft insertion fabric, a non-woven fabric and a woven fabric.

In some embodiments, the article may also include absorbent pad juxtaposed with and secured to one of the first and second exterior surfaces of the article, and covering less than one-third of the one surface. The absorbent pad may include hydrophilic foam, which may include a plurality of open cells and may include at least one of polyurethane, silicone, polyethylene, and gauze. At least one of an adhesive, a cohesive, and a web adhesive may be used to secure the pad to the surface.

Under another aspect, a method of making a cohesive article having oppositely-facing first and second surfaces includes providing a foam layer; providing a second layer in juxtaposition with the foam layer; and applying a cohesive composition to at least a portion of at least one surface of the foam layer, and optionally to at least a portion of at least one surface of the second layer. The cohesive composition may be applied such that the composition permeates the thickness of the article and secures the second layer to the foam layer. The method may include winding the article into a roll such that the first surface is cohesively attached to the second surface. The second layer may include at least one of a second foam layer, an elastic layer, a warp-knit fabric, a warp-knit weft-insertion fabric, an elastic fabric, a knit fabric, a woven fabric, and a non-woven fabric.

Under another aspect, a medical bandage includes an elastically extensible article having first and second oppositely-facing exterior surfaces each of which is coated with a cohesive composition; and an absorbent pad juxtaposed with and secured to one of the exterior surfaces of the extensible article. The pad can be placed over a wound, sore, or ulcer, and the remainder of the cohesive tape or bandage wrapped around the afflicted body part, for example twice or more. The elastically expansible tape or bandage compresses the pad against the wound both securely and comfortably. Depending on the application, the foam pad is preferably hydrophilic foam, and provides “wicking” of fluids, such as wound exudate, with which it is in contact.

In some preferred embodiments, the oppositely facing surfaces of the tape or bandage to which the pad is attached are each coated with a cohesive composition, the pad has a thickness greater than about 0.1 inches and is sized and adapted to cover a wound on a body part, and the longer elastically expansible tape or bandage is sized to wrap several times around the body part. The product may be provided as a roll in which the cohesive coated surfaces contact and cohesively adhere to each other, and may be in sterile condition in a sealed package. Typically, the product is intended for a single use and includes only a single pad.

In some embodiments, the absorbent pad has a thickness not less than three times the thickness of the elastically extensible article and covering less than one-third of the surface of the article to which it is secured. The absorbent pad is sized and adapted to cover a wound on a body part, e.g., the absorbent pad has a length less than that of the circumference of the body part. The article may have a length at least twice the circumference of the body part. The absorbent pad can include hydrophilic foam, which may include a plurality of open cells. The absorbent pad may include at least one of polyurethane, silicone, polyethylene, and gauze. At least one of an adhesive, a cohesive, and a web adhesive may be used to secure the pad to the surface. A package may have the medical bandage sealed therein, and the medical bandage may be in a sterile condition. The bandage may be wound into a roll such that the first surface is cohesively attached to the second surface, and the bandage may be in a package, and optionally in a sterile condition therein. In some embodiments the medical bandage has only one absorbent pad.

As used herein, the term “article” encompasses tapes and bandages, as well as other structures that are used to perform functions similar to those of tapes and bandages in medical, sports, and other applications. Further as used herein, the term “about” means approximately, in the region of, roughly, or around. When the term “about” is used in conjunction with a numerical value or range expressed as a whole number, it modifies that value or range by extending the boundaries above and below the numerical value(s) set forth to modify the numerical value(s) by plus or minus 10% of the stated value, rounded to the nearest whole number. When the term “about” is used in conjunction with a numerical value or range expressed as a decimal, e.g., 0.01, it modifies that value or range by extending the boundaries above and below the numerical value(s) set forth to modify the numerical values to encompass values that round to the expressed decimal value, e.g., the numerical value 0.1 encompasses values extending from 0.051 to 0.149, and the numerical value 0.25 encompasses values extending from 0.0246 through 0.0254.

### BRIEF DESCRIPTION OF THE FIGURES

FIG. 1 is a top view, partially broken away of a tape or bandage having an upper backing layer of woven warp-knit weft insertion fabric 12, a bottom foam layer 14 of open cell polyurethane foam, and a middle layer of longitudinally-extending, transversely spaced (approximately 12 per inch) elastic strands 16, according to one embodiment of the invention. The laminated structure is laminated together with a cohesive composition that permeates all three layers.

FIG. 2 is a cross-sectional view at line 2-2 of the embodiment of FIG. 1, showing the cohesive composition 18 impregnating all three layers.

FIG. 3 is a depiction of an apparatus and process for fabricating a cohesive foam tape, according to one or more embodiments of the invention. In the illustrated embodiment, a foam base fabric and a warp knit backing (Milliken 18×18, 30×70) are coated with a cohesive formulation and laminated together on either side of stretched elastic yarns to produce a laminated elastic article that is further heat cured and formed into a roll.

FIG. 4 is a top view of a foam layer cohesive article having foam wound care pad for direct application to an open wound, according to one embodiment of the invention. The foam wound care pad 20 is attached to the foam layer elastic cohesive bandage 30, for example attached with an adhesive agent, and can be applied directly to an open wound or ulcer and secured in place by winding the foam layer cohesive bandage securely around the affected area.

FIG. 5 is a cross-sectional view at line 3-3 of FIG. 4 showing the foam wound care pad 20 attached to the foam layer 24 of the foam layer cohesive bandage 30 with a standard web adhesive 25. The cohesive agent 28 permeates the 18×18 warp knit backing 22, the elastic yarn layer 26 (spandex yarns extending longitudinally), and the thin foam layer 24 of the foam layer cohesive bandage structure 30.

FIG. 6 is a microscope image of an uncoated foam layer having a thickness of about 0.025 inches.

FIG. 7 is a microscope image of the foam layer side of an article having a foam layer with thickness of about 0.025 inches, an 18×18 warp-knit (weft-insertion) fabric, and a latex-free cohesive composition according to one embodiment of the invention.

FIG. 8 is a microscope image of the foam layer side of the article of FIG. 7, the foam layer being compressed onto a glass slide (image taken through the glass slide).

FIG. 9 is a microscope image of the foam layer side of the article of FIG. 7, the foam layer side being compressed onto a glass slide (image taken through the glass slide).

FIG. 10 is a microscope image of the warp-knit (weft-insertion) fabric side of the article of FIG. 7, the foam layer side being compressed onto a glass slide.

### DETAILED DESCRIPTION

Overview

In general, the invention provides foam layer cohesive articles, for example medical bandages and wraps. In some aspects, the foam layer cohesive articles include a foam layer, and optionally include one or more additional layers, such as an elastic layer, or a fabric, which can provide enhanced elasticity, strength, softness and/or cohesion (more below). The articles typically have first and second oppositely-facing exterior surfaces, and in some aspects both of these first and second surfaces are at least partially coated with a cohesive composition. In various embodiments the cohesive composition substantially permeates the foam and secures the foam layer to other layers within the article; however in general the cohesive composition need not permeate the foam or other layers, but simply coat at least a portion of one or both of the major exterior surfaces of the article. The article may also include a foam pad that can be applied to a wound, as discussed in greater detail below. The article can be wound upon itself to form front to back oriented layers.

The presence of the foam layer is useful in many respects. For example, if the article is used as a wrap, the foam layer provides enhanced comfort and softness relative to bandages that do not include a foam layer. In addition, in embodiments where the foam layer defines at least a portion of one of the major exterior surfaces of the article, the microscopic structure of the foam can enhance the cohesive properties of the article. For example, the foam layer may include a plurality of open cells that have surfaces facing the exterior of the article, and the cohesive composition may coat these open cell surfaces without filling the cells. As shown in FIGS. 6-10, open cells appear to essentially form tiny, outward-facing “suction cups.” If these suction cups are compressed against a surface, e.g., against another surface of the article if the article is wound around a body part, or against a non-porous surface of a medical device being affixed to a body part, the suction cups are believed to form a partial vacuum that imparts a particularly secure cohesive property to the article. It has been observed that if the article gets wet while it is wrapped around a body part, it does not unravel as conventional latex free cohesive bandages would, but rather maintains the secure fit around the body part.

The foam layer need not define the entirety of one of the major exterior surfaces of the article in order to provide the article with enhanced cohesion. For example, as discussed in greater detail below, a porous fabric (such as a woven scrim, among others) can be applied over the foam layer. This fabric may be porous enough that the foam layer is exposed through the fabric, as shown in FIG. 10. Without wishing to be bound by theory, it appears that this allows, thus at least some of the exposed open cells on the surface of the foam layer, in conjunction with the fabric coated layer, to behave as tiny suction cups when the fabric-coated foam layer is compressed against a surface, and thus maintaining at least some of the enhanced cohesion of the article. While in some embodiments the presence of the porous fabric may reduce the enhancement in cohesion as compared to a fabric-free embodiment, however the porous fabric may provide other useful features such as enhancing the strength of the article, allowing the article to be more uniformly torn by hand, and/or providing a desired hand-feel to the article.

The foam layer may also include at least some closed cells, or even have a substantially entirely closed-cell structure. The closed cells will not necessarily provide a comparable “suction cup” action to the open cells, but the foam will still impart a soft feel to the article.

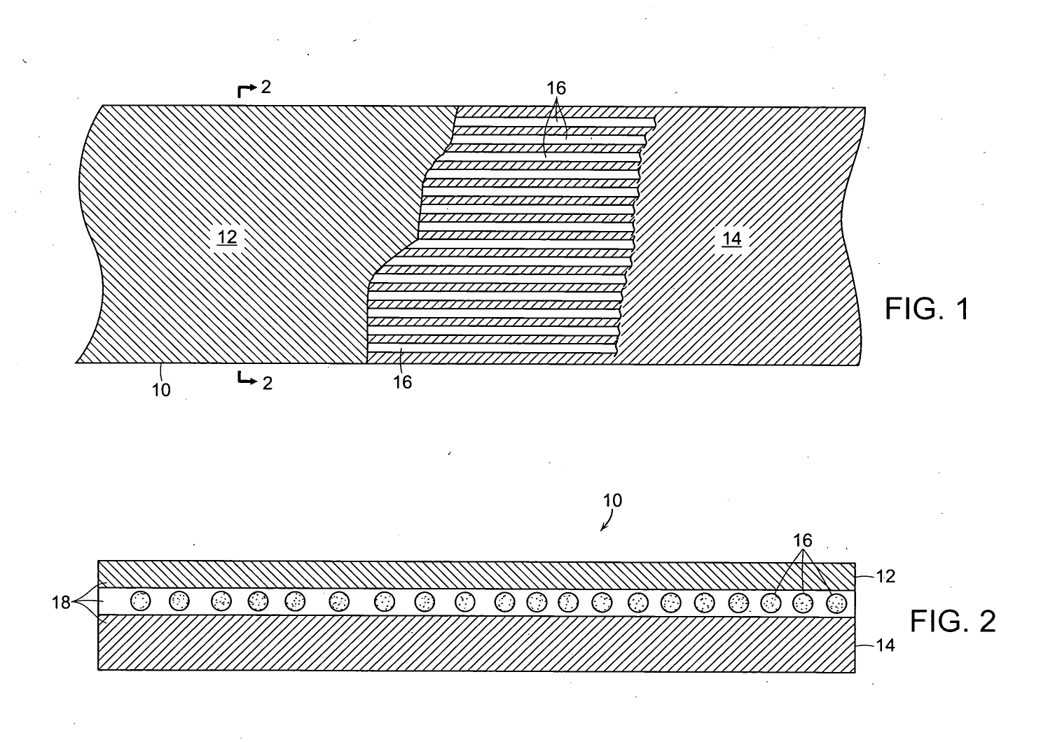

FIGS. 1 and 2 depict one illustrative embodiment of a foam layer cohesive article, generally designated 10. In this embodiment, the article includes a backing layer 12 of warp-knitted (weft-insertion) fabric, a bottom layer 14 of polyurethane foam, and a middle layer of longitudinally-extending, transversely spaced (e.g., about 12 per inch) elastic strands 16. The three-layer structure is laminated together with a cohesive composition 18 that impregnates all three layers. The cohesive composition substantially coats the major exterior surfaces of the article, and also permeates all three layers, thus securing them to each other.

In use, the foam layer 14 is inherently elastic, that is, it can be deformed extensively and then substantially return to its original shape. Thus, the presence of the elastic strands 16 is not necessary in some embodiments. However in certain applications the presence of the elastic may enhance the compression the article can exert if, for example, the article is wound around a body part; the elastic may also add strength to the wrap, and more rapid recovery of the article to its original shape after stretching.

Although the embodiment of FIGS. 1 and 2 includes a warp-knit (weft-insertion) fabric in the backing layer 12, in general a variety of different layers (or no layer at all) can be used in the backing. The backing layer 12 may include a plurality of layers, For example, the backing can include an elastic fabric, which may include elastic yarns woven throughout the fabric; in this case a separate elastic layer 16 may not be necessary, although it can still be included if desired depending on the application. The backing layer can also include a non-woven fabric. For example, embodiments in which an open-cell foam layer defines a first major exterior surface of the article, and in which a non-woven fabric is used as a backing and thus defines a second major exterior surface of the article, have been found to be particularly cohesive when the foam layer is compressed against the non-woven fabric backing. Without wishing to be bound by theory, it is believed that the non-woven fabric backing may provide an enhanced surface area relative to some other kinds of fabrics, and/or (as discussed above) may be sufficiently open that some of the open-cell “suction cups” of the foam layer underlying the non-woven fabric are available for use. Knit fabrics can also be used in the backing layer 12, for example chain knits, circular knits, or a warp-knit (weft-insertion) fabric as shown in the illustrated embodiment. Woven fabrics can be used, such as a woven scrim or an open mesh fabric. In some embodiments, one or more of the layers used in the backing is substantially porous, for example has about a 25% to 75% open structure, e.g., about 50% open.

The layer(s) used in the backing layer 12 are not limited to fabric-based layers. For example, in some embodiments, a second foam layer is used in the backing. The second foam layer can provide enhanced comfort, as well as a stronger peel strength. This can result in an enhanced grip, for example if the article is used on a hand.

As mentioned above, not all embodiments will include backing and/or elastic layers, as the foam layer itself provides many useful properties, such as cohesion, softness, and strength, when coated with the cohesive composition in the absence of other layers.

Also, as mentioned above, an additional layer, e.g., a fabric layer can also be added to the front of the foam, e.g., in addition to a backing layer added to the back of the foam. The front additional layer can include the fabrics mentioned above and/or can include an elastic layer. The backing and/or front fabrics can also have different strengths in the machine and cross directions so as to provide facile and even hand-tear to the foam layer cohesive article.

Although the discussion above, and FIGS. 1 and 2 treat the elastic layer 16 as being “separate” from the backing, the backing can also be considered to be both the fabric layer and the elastic layer together. The term “backing” or “second layer” should not be construed as being limited to a single-ply layer, but in fact can be multiple-ply and have many layers. The backing can be secured to the foam layer using the cohesive composition, for example by permeating the foam and the backing with the cohesive composition which binds the layers together when it dries.

Some combinations of layers that can be used to form various embodiments of foam layer cohesive articles are listed below. The first listed layer in the embodiment defines at least a portion of the first major exterior surface of the article, the last listed layer defines at least a portion of the second major exterior surface of the article, and any layers in between are present in the order listed and may themselves define at least a portion of the first and/or second major surfaces of the article, depending on the porosity of any intervening layers. The listed embodiments are not intended to be limiting, or inclusive of all possible embodiments.

Warp-knit (weft-insertion) fabric layer, elastic layer, foam layer.

Warp-knit (weft-insertion) fabric layer that is precoated with color, elastic layer, foam layer.

Warp-knit (weft-insertion) fabric layer, foam layer.

Warp-knit (weft-insertion) fabric layer, foam layer, warp-knit (weft-insertion) fabric layer.

Warp-knit (weft-insertion) fabric layer, elastic layer, foam layer, warp-knit (weft-insertion) fabric layer.

Warp-knit (weft-insertion) fabric layer, elastic layer, non-woven fabric layer, foam layer.

Foam layer, warp-knit (weft-insertion) fabric layer, elastic layer, foam layer.

Foam layer, elastic layer, non-woven fabric layer.

Foam layer, elastic layer, foam layer.

Foam layer, elastic layer.

Foam layer, warp-knit (weft-insertion) fabric layer, foam layer.

Foam layer, elastic layer, woven fabric layer, foam layer.

Foam.

Non-woven fabric layer, elastic layer, foam layer.

Non-woven fabric layer, woven fabric layer, elastic layer, foam layer.

Open mesh fabric layer, foam.

Specific details of different kinds of useful fabrics, elastic layers, cohesive compositions, foam layers, and the like can be found below. Additionally, those skilled in the art will recognize that other layers and compositions can be used.

The article may be wound into a roll. In some embodiments, the first major exterior surface of the article is wound onto and cohesively attaches to the second major exterior surface of the article, or vice versa. In other embodiments a removable release layer is placed in between the major exterior surfaces of the article. The release layer is not cohesive, but readily detaches from the major exterior surfaces of the article. A release layer may be useful in circumstances where cohesion between the major exterior surfaces of the article is relatively high, and the presence of the release layer would facilitate unwinding of the rolled article or otherwise facilitate use of the article. Note that the article need not be rolled in order to use a release layer.

Characteristics of Exemplary Embodiments of Foam Layer Cohesive Articles

In many embodiments, the foam layer cohesive articles provide secure cohesive bonds, for example when the front foam layer is bonded to the backing layer back of the article, e.g., when the article is wound upon itself to form front to back oriented layers, either on the roll or if it is used to wrap a body part. The strength of this secure cohesive bond between front to back oriented layers of the article can be characterized by a peel force bond strength of, for example, between about 5 oz/in-w and about 40 oz/in-w as measured in a standard peel force test, depending on the particular application and configuration, e.g., ratio of open cells to closed cells in the foam, the presence of additional layers, and the cohesive composition. In some embodiments, this peel bond force strength may be between about 12 oz/in-w and about 35 oz/in-w, between about 20 oz/in-w and about 30 oz/in-w, or about 25 oz/in-w in a standard peel force test.

In some embodiments, the secure cohesive bond provided by the foam layer front of the article is further characterized by a shear force bond strength of 2 lb/in² to 30 lb/in in² a standard shear force strength test to a stand surface substrate, depending on the particular application and configuration as mentioned above. In some embodiments, the article may have a shear force bond strength between about 5 lb/in² and about 20 lb/in², or between about 9 lb/in² and about 15 lb/in², or between about 11 lb/in² and about 13 lb/in², or about 12 lb/in² in a standard shear force strength test.

In some embodiments, the overall laminated elastic article is characterized by the ability to stretch 50% to 200% beyond its original unstretched length before it fails. The inherent elasticity of the foam and of other layers that may be present determine, in part, the article's ability to stretch before failure. For example, the presence of a fabric (e.g., a warp-knit (weft-insertion) fabric may prevent the article from stretching as far as it otherwise would be able to, because the yarns of the fabric may not themselves be extensible. Thus the weave of the fabric may limit the extensibility of the article. As discussed below, the fabric may be “gathered” during fabrication so that the article is extensible to a desired percent stretch before reaching the maximum extension of the fabric, at which point further stretch would at least partially damage the article. In particular embodiments, the article has a percent stretch of 100% to 180%, or about 120% to about 160%, or about 140% beyond the unstretched length before failure.

In some embodiments, the overall article may be further characterized by having a tensile strength of 8 lb/inch to 25 lb/inch in a standard tensile strength test, for example about 12 lb/inch in a standard tensile strength test. The overall article may be further characterized, in many embodiments, by a weight of from 30 g to 100 grams per square meter of the overall article, with the cohesive composition making up about 20 to 70% of this overall weight of the article. In certain embodiments, the article has a weight of from 40 to 80 grams per square meter of the overall article, with the cohesive composition making up about 25 to 45% of this overall weight of the article. In one embodiment, the article has a weight of about 60 g/m² and the cohesive composition comprises about 35% of the weight of the article.

Apparatus and Methods of Making Foam Layer Cohesive Articles

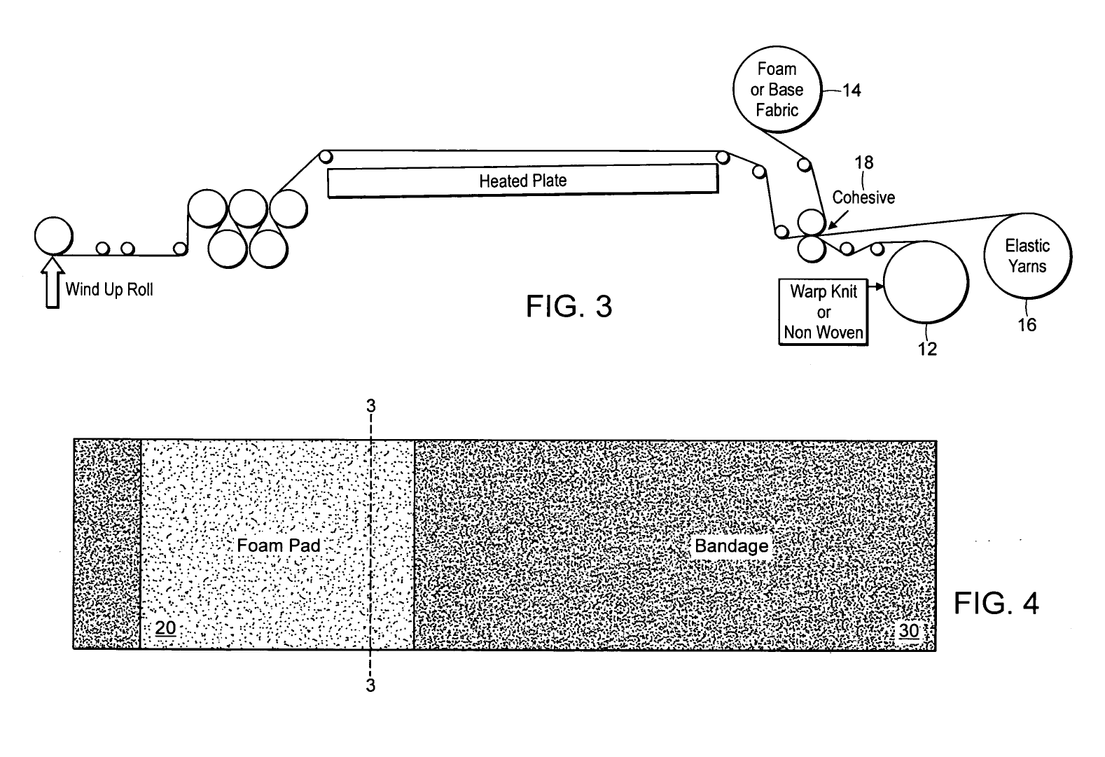

An exemplary apparatus for preparing one embodiment of a foam layer cohesive article is shown schematically in FIG. 3. The apparatus includes three separate feed rolls for supplying a foam layer 14, warp-knit (weft-insertion) fabric backing layer 12, and an elastic layer 16, e.g., elastic yarns. The elastic layer 16 is fed between the foam layer 14 and the warp-knit fabric backing layer 12. The foam layer 14, the warp-knit fabric backing layer 12, and the elastic layer 16 are guided together into nip rolls that supply a metered amount of a cohesive composition 18 to the layers from a reservoir. As described in greater detail below, the cohesive composition can be, e.g., a synthetic, water based cohesive formulation or a natural rubber based cohesive formulation. In many embodiments, the cohesive formulation is of a solids content and viscosity that permits impregnation and coating of the foam base and warp knit fabric backing layers of the article. Although the actual composition may vary depending upon the particular cohesive and foam and backing layers used, some exemplary cohesive formulations contain about 30 to 65 wt % solids. Additives, e.g., antifoaming agent, can be added to improve the machinability of the cohesive formulation.

In certain embodiments, the backing layer 12 is fully extended and the foam and the elastic layer are stretched when they are laminated together with the cohesive composition. For example, the elastic layer may be stretched by about 50 to about 250%, or about 130 to about 170%, or about 150% of its original unstretched length when it is laminated to the backing layer and the foam layer. The foam layer may be stretched by about 0% to about 20% when it is laminated to the elastic layer and the backing layer, or may be fully extended (but not stretched) when it is laminated to the elastic layer and the permeated backing layer. After passing through the nip rolls, which supply compression to the layered article, the layers may be further laminated together by passing between an infrared heater and a heated plate maintained at an appropriate temperature. The heater can be heated air, heat lamps, or any other conventional source of heat. The laminate structure then is passed through multiple rollers to dry the laminated structure and secure the warp knit fabric backing to foam layer front of the article. In many embodiments, essentially all of the carrier liquid is removed in the drying step, and the finished product is then wound into a take-up roll. The take-up roll can then be used directly or rewound into a finished roll of any desired length, width and winding tension.

Note that different embodiments of the foam layer cohesive articles can be fabricated using modifications of the apparatus depicted in FIG. 3, or with entirely different machinery and/or methods. For example, if the article does not include a backing layer and/or elastic layer, those reels and steps can be omitted. Or, for example, the backing layer and/or the foam layer can be precoated with the cohesive composition, and the elastic layer positioned between the precoated woven backing layer and the pre-coated foam layer. The backing layer precoated with the cohesive composition may include a fabric, e.g., a woven material, permeated with a binder (such as acrylic nitrile) and then coated with a cohesive agent (such as natural rubber or neoprene latex).

In one illustrative embodiment, a foam layer cohesive article for use, e.g., as a tape or a bandage, can be fabricated by permeating a backing layer of warp-knit, weft-insertion polyester fabric with a cohesive composition that has about 60% dry weight natural rubber or neoprene latex and about 33% dry weight rosin ester tackifying agent(s). The cohesive composition is also used to permeate a foam layer of open cell polyurethane foam material having a density of about 1.40 lb/ft³, a thickness of about 0.025 inches, and weight of about 22 grams/m². The cohesive-permeated backing layer is then laminated to the cohesive-permeated foam layer along with an elastic layer that is positioned between the permeated backing layer and the permeated foam layer. The elastic layer laminated between the permeated backing layer and the permeated foam layer is made up of elastic spandex yarns having a denier of about 210, a percent stretch of 700 to 800% beyond their unstretched length before failure, and a weight of about 6.5 grams per square meter of the overall article. Finally, the resulting laminated article is dried to produce a foam layer cohesive article that can be formed into a roll or used directly.

Cohesive Compositions

The cohesive composition may be any suitable natural rubber or synthetic cohesive formulation. Natural rubber latex is inherently cohesive, meaning that it sticks to itself rather than to other material. Suitable cohesive natural rubber latex cohesive formulations are known in the art. Alternately, the cohesive composition can be latex free, which allows an article coated with the composition to be used with patients who have latex allergies.

Some useful synthetic cohesive formulations are described in U.S. Pat. No. 6,154,424. In some embodiments, these synthetic cohesive formulations include a synthetic, water-based and inherently crystalline elastomer, rather than natural rubber latex, in combination with an amount of one or more tackifying agents that disrupts the inherently crystalline structure of the elastomer and maintains the elastomer in a partial polycrystalline state. Suitable inherently crystalline elastomers for use in formulating these natural rubber-free synthetic cohesive formulations include water-based polychloroprene emulsions such as poly-2-chloro, 1-4 butadiene and certain water based polyurethanes, that are inherently capable of crystallization, i.e., polyester polyurethane and polycaprolactone polyurethane. Or, for example, the inherently crystalline, water-based, synthetic elastomer can be a polychloroprene, such as DuPont NEOPRENE LTX-654 (or NEOPRENE LTX-671A). Polychloroprene is particularly useful in latex-free embodiments of the article.

In some embodiments, the synthetic cohesive formulations also include one or more tackifying agents, which may disrupt the crystallinity of the inherently crystalline elastomeric polymer(s) and thus bring them to, and arrest them in, a structure with a desired level of partial polycrystallinity, and thus exhibits a desired cohesiveness. In some respects, such a composition behaves similarly to natural rubber latex, exhibiting a cohesive property when the degree of partial polycrystallinity is maintained within a range (typically determined empirically) between a completely amorphous state and a highly crystalline state.

As used in the present invention, the terms “tackifier” and “tackifying agent” herein refer to a class of thermoplastic polymers used to affect the characteristics of a finished polymeric product and includes the tackifying resins listed above, naturally occurring rosins, rosin esters, and plasticizers.

In some embodiments, the tackifiers used to produce cohesive forms of these synthetic elastomeric materials are of the same type used in connection with natural rubber, although the amount(s) of any particular tackifier(s) used to form a stable cohesive will vary within empirically defined limits. Exceeding the limits produces either a non-cohesive or an amorphous pressure-sensitive adhesive, neither of which is useful as a synthetic cohesive agent. In some embodiments, the tackifiers used to arrest the elastomer in the desired polycrystalline state are one or more of a rosin ester derivative, a petroleum derivative, a hydrocarbon resin, an acrylic polymer, a butadiene-based polymer or a combination of one or more types such as rosin ester/hydrocarbon resin. In one illustrative embodiment, the tackifying agent is PERMATAC H7120 and/or AQUATAC 6085.

The term “rosin” as used herein refers to a naturally occurring material extracted from stumps of pine trees whose principal component is abietic acid. The term “rosin ester” as used herein, refers to the carboxyl group of abietic acid which has been esterified with aromatic and aliphatic alcohols. The term “hydrocarbon resins” as used herein refers to lower-molecular-weight thermoplastic polymers derived from cracked petroleum distillates, terpene fractions, coal tar, and a variety of pure monomers. Although a single tackifying resin can be used, blends of two or more with different melting points (and molecular weights) have been found to produce cohesive products with better final properties. In some circumstances, plasticizers may be used in lieu of one or more tackifier resins. Synthetic elastomers such as polychloroprene, e.g., NEOPRENE LTX-654, and tackifying agents are commercially available in dispersion and emulsion forms.

When compounding the elastomer and tackifiers, there typically exists for each elastomer a “window” of compounding in which the structure of the polychloroprene or other elastomer is crystalline, and within which the degree of crystallinity can be modified so that the material has cohesive properties. The extent of the “window” varies depending on the particular elastomer, and is determined empirically. At one extreme of the “window”, the elastomer becomes non-cohesive, and at the other extreme, it becomes pressure-sensitive. The state of the material within its “window” depends on the extent to which the polycrystalline structure of the polychloroprene or other elastomer is disrupted, and can be varied using different amounts and types of tackifying agents. For any particularly water-based inherently crystalline, synthetic elastomer, the amount and type of tackifier required to arrest the elastomer in a partially crystalline, cohesive state is empirically determined, using tackifiers and protocols similar to those long employed in the production of cohesive natural rubber latex materials and known to one of skill in the art. The cohesive composition can include one or more elastomers such as natural rubber latex, synthetic rubber latex, polyisoprene, polychloroprene, polyester polyurethane, and polycaprolactone polyurethane. The composition can also include at least one tackifying agent in an amount effective to disrupt the crystalline structure of the elastomer and maintain the elastomer in a partial polycrystalline state such that the elastomer possesses a cohesive property. A particularly useful cohesive composition of the invention is an anionic colloidal aqueous dispersion of the inherently crystalline elastomer polychloroprene. In certain embodiments, the inherently crystalline elastomer is a neoprene latex. In general, the neoprene latex makes up 50% to 70% of the dry weight of the cohesive composition permeating the article. In particular embodiments, the neoprene latex makes up about 60% of the dry weight of the cohesive composition permeating the article. The neoprene latex can be, e.g., NEOPRENE LTX-654 or NEOPRENE LTX-671A (Dupont, Wilmington, Del.).

The tackifying agent(s) used in the synthetic water-based cohesive formulation may be an aqueous rosin ester dispersion (e.g., PERMATAC H7120 (Neville Chemical Company, Pittsburgh, Pa.) and/or AQUATAC 6085 (Arizona Chemical, Jacksonville, Fla.)). In particular embodiments, the tackifying agent(s) comprise about 33% of the dry weight of the cohesive composition permeating the article.

The cohesive composition may include about 30% to 65% solids when the composition is permeated into the foam layer (i.e., before drying). In particular embodiments, the cohesive composition is a synthetic cohesive that includes 35% to 50% solids, or about 40% solids. In some embodiments, the cohesive composition component of the overall laminated elastic article contributes about 10 g/m² to 70 g/m² of the overall article's weight, for example about 15 g/m² to 35 g/m², or about 25 g/m² of the overall article.

Foam Layer

In some embodiments, the foam layer is a cellular sheet material formed of a suitable material, such as chemically foamed or aerated plastic material, foamed rubber or a non-hardening cellulose sponge material. In some embodiments, the foam layer includes a plurality of open cells which behave as tiny “suction cups” that enhance the cohesiveness of the article. These open cells may define at least a portion of one of the major exterior surfaces of the article. In some embodiments, the foam layer includes a plurality of closed cells. The closed cells do not necessarily provide as strong a “suction cup” effect as open cells would, however the closed cells do provide enhanced cohesion and comfort relative to a foam-free product. The cohesion of the article, as well as the adhesion of the article to other surfaces (such as the non-porous surfaces of braces or other medical equipment) can be adjusted by, among other things, selecting the ratio of open cells to closed cells in the article, as well as adjusting the cohesive composition appropriately.

Open cell foams and closed cell foams are well known in the art, and those of ordinary skill in the art will recognize that foams termed “open cell” will naturally include some closed cells, and that foams termed “closed cell” will naturally include some open cells. Thus the terms “open cell” and “closed cell” do not imply that the foam must necessarily include 100% open or 100% closed cells. In general, in closed cell foams most of the cells are closed off from each other, and water absorption is low. Open-cell foams have an interconnecting cell structure, absorb liquids, are generally softer than closed-cell foams, and have less structural integrity than open cell foams.

In some embodiments, the foam material includes one or more of polyurethane, polyester, polyester/polyurethane and polyethylene. When incorporated into the article, the layer may have a weight of from 18 to 30 grams per square meter of the article. In particular embodiments, the foam layer has a weight about 22 grams/m² of the article. When constructed of polyurethane, the foam layer generally has a density of 1.00 lb/ft³ to 3.00 lb/ft³, e.g., about 1.40 lb/ft³. The foam layer may have a thickness between about 0.01 inch to about 0.25 inch, for example between about 0.025 inch and 0.035 inch. The foam layer may be of any thickness desired for a particular application. In general the greater the thickness, the greater the cushioning effect; however a greater thickness also increases the bulk of the article so the appropriate thickness will depend on the particular use. For example, a thinner foam may be useful for arm or leg wounds in which clothes would be worn over the wrapped article, whereas a thicker foam may be useful where applied over a bruise since it would provide more cushioning, or for use with animals in which case the wrapped article would be likely to experience additional wear.

In some embodiments, the foam layer is a thin-gauge sheet of polyurethane or polyester/polyurethane foam material having a thickness on the order of 0.025 inches. One suitable polyester polyurethane foam sheeting material type is manufactured and sold by W.T. Burnett & Co. (Jessup, Md.) under the product number S82F polyester polyurethane foam. This foam sheeting material has a density of about 1.4±10% lbs./ft³, a minimum tensile strength of 22.0 psi and an average tensile strength of 30.0 psi, a minimum tear resistance of 3.00 pli and an average tear resistance of 4.00 pli, and a minimum elongation of 300% (average of 400%) (as determined by using the ASTM-D3574 standard methods of testing flexible cellular materials-slab, bonded and molded urethane foam). The S82F polyester polyurethane foam further has a minimum compression force deflection of 0.35 psi and an average compression force of 0.50 psi at 25% deflection and, at 50% deflection, a minimum compression force deflection of 0.40 psi and an average compression force of 0.55 psi at 25% deflection. The S82F polyester polyurethane foam having a thickness of 0.025 inches produces a laminated article with satisfactory cohesive and cushioning properties, however other thicknesses (e.g., up to 0.0 inch or even greater) may be employed to provide additional cushioning.

Other exemplary materials suitable for use as a foam layer include a flexible foamed polyester material, which may provide enhanced flame resistance. Or, foamed rubber sheeting or non-hardening cellulose sponge sheeting may be employed as the core, either in combination with or in substitution for sheeting of foamed plastics material. The foam layer may alternately be a sheet of a suitable foamed thermosetting material, or foamed rubber sheeting, or, non-hardening cellulose sponge sheeting. Additionally, the foamed material may incorporate fire retardant or suppressant agents, which may be selected so as to resist leaching during normal wear or exposure to the elements to which the article is likely to be subjected.

In some embodiments, the foam layer is fabricated or commercially purchased with a plurality of open cells on at least one of its major surfaces. At least some of the open cells remain open during fabrication of the article, even after permeation with the cohesive composition and lamination to other layer(s). The open cells then act as “suction cups” and thus enhance the cohesiveness of the article. In other embodiments, the foam layer is fabricated or commercially purchased with a plurality of closed cells. During the lamination operation a number of the closed cells may be partially severed and opened. In some embodiments, the foam layer as fabricated or purchased has a cell size of the individual cells that is maintained below a determined maximum, and, a preponderance of the cells are of smaller size and extent than the size of the largest of the cells.

Front and/or Backing Layers

As noted above, the front and/or backing layers of the article may include a second foam layer, an elastic layer, an elastic fabric, a knit fabric, a woven fabric, or a nonwoven fabric, among other things. Although the majority of the description in this section is directed to backing layers, it should be noted that it applies equally to front layers, applied to the other side of the foam layer.

In some embodiments, the backing layer 12 may include one or more layers that facilitate hand-tearing of the article 10, and/or provide article 10 with suitable longitudinal tensile strength for use in applications such as, for example, wrapping a limb or other body part, or any other suitable application.

In some embodiments the backing layer may be a warp-knit weft-insertion fabric. In particular, in a warp-knit weft-insertion fabric, the warp yarns may include a plurality of longitudinally-spaced knitted loops through which the weft yarns extend transversely of the article. The warp yarn(s) may be of lower tensile strength than the weft yarn(s) so as to facilitate hand tear, but the relative strengths of the overall article in the machine direction versus the cross direction is also influenced by the density of the warp and weft yarns as described in further detail below. Accordingly, the overall strength of the article in the machine direction may be higher than that in the cross direction, despite the fact that a weft yarn having a higher denier than that of the warp yarn is utilized.

The warp yarns and weft yarns of the warp-knit weft-insertion fabric may be yarns of any suitable material. For example, the warp yarns and weft yarns may be yarns of polyolefin, polyester, polycotton, cotton, or any other suitable material that allows for hand-tearing of tape and provides the desired tensile strength. The weft yarns extending transversely of the article may be, for example, texturized filament yarns.

The warp yarns of the warp-knit weft-insertion backing layer may be spaced at a density in the range of 9 to 48 yarns per inch as measured transversely of the article 10. In some embodiments, the warp yarns may be spaced at a density in the range of 12 to 24 yarns per inch, particularly at a density of about 18 yarns per inch. Alternatively, the warp yarns may be spaced at a density in the range of 18 to 30 yarns per inch, 30 to 48 yarns per inch, or any other suitable range of densities. The warp yarns of a warp-knit weft-insertion backing layer 12 may have a denier in the range of 20 to 80. In some embodiments, the warp yarns may have a denier in the range of about 30. Alternatively, the warp yarns may have a denier in the range of 20 to 60, 40 to 80, 60 to 100, or any other suitable range of deniers.

The weft yarns of the warp-knit weft-insertion backing layer may be spaced at a density in the range of 6 to 48 yarns per inch as measured longitudinally of the article 10. In some embodiments, the weft yarns may be spaced at a density in the range of 9 to 18 yarns per inch as measured longitudinally of the article, particularly at a density of about 12 yarns per inch. Alternatively, the weft yarns may be spaced at a density in the range of 6 to 24 yarns per inch, 18 to 36 yarns per inch, 30 to 48 yarns per inch, or any other suitable range of densities. The weft yarns of the warp-knit weft-insertion backing layer 12 may have a denier in the range of 50 to 200. In some embodiments, the weft yarns may have a denier in the range of 60 to 100, particularly a denier of about 70. Alternatively, the weft yarns may have a denier in the range of 40 to 170, 170 to 300, or any other suitable range of deniers.

In some embodiments, the warp-knit weft-insertion backing layer may have a weight of not more than about 50 grams per square meter. In some embodiments, the warp-knit weft-insertion backing layer may have a weight in the range of 10 to 20 grams per square meter, particularly about 15 grams per square meter. Alternatively, the warp-knit weft-insertion backing layer may have a weight in the range of 10 to 30 grams per square meter, 20 to 50 grams per square meter, or any other suitable range of weights.

An example of an illustrative fabric that may be used for the warp-knit weft-insertion fabric is style number 071355 obtained from Milliken & Company of Spartanburg, S.C. (“the 18×12 Milliken fabric”). The Milliken fabric is a polyester warp-knitted weft-insertion fabric having a warp denier of about 30 and a weft denier of about 70. The Milliken fabric weighs approximately 0.33 ounces per square yard, has warp yarns spaced at about 18 yarns per inch and weft yarns spaced at about 12 yarns per inch, and has a tensile strength of about II pounds per inch (machine direction).

Another example of an illustrative fabric that may be used is an 18×18 warp-knit weft insertion fabric, style number 997590 (pattern # 550) obtained from Milliken & Company of Spartanburg, S.C. (“the 18×18 Milliken fabric”). This Milliken fabric is a polyester warp-knitted weft-insertion fabric having a warp denier of about 30 and a weft denier of about 70. The Milliken fabric weighs approximately 14.4 grams per square meter, has warp yarns spaced at about 18 yarns per inch and weft yarns spaced at about 18 yarns per inch, and has a tensile strength of about 11 pounds per inch (machine direction).

Another exemplary warp-knit weft-insertion fabric is style number J477 obtained from Chima, Inc., of Reading, Pa. (“Chima fabric”). The Chima fabric is a polyester warp-knitted weft-insertion fabric having a warp denier of about 50 and a weft denier of about 150. The Chima fabric weighs approximately 0.74 ounces per square yard, and has a tensile strength of about 22 pounds per inch.

The backing layer of the article may also include a woven scrim fabric. A “scrim” fabric is a loose plain-woven fabric, frequently of cotton, with fine to coarse mesh. Scrim woven fabrics also have warp (machine direction) yarns and weft (cross direction) yarns, with adjacent warp yarns extending longitudinally on opposing sides of the plane defined by weft yarns in a non-looped fashion. An example of an illustrative scrim fabric is style number 013228400011 obtained from DeRoyal Textiles of Camden, S.C. (“DeRoyal fabric”). The DeRoyal fabric is a cotton scrim woven fabric having a warp yarn density of about 32 yarns per inch measured transversely of the tape and a weft yarn density of about 28 yarns per inch measured longitudinally of the tape. The DeRoyal fabric weighs approximately 1.31 ounces per square yard. Still other examples of fabrics that may be used for the warp-knit weft-insertion backing layer includes greige cloth and other such scrim woven fabrics known in the art.

The backing layer may include a nonwoven layer of material. The fibers of a nonwoven material are intimately entangled with each other to form a coherent, breathable fibrous material. Nonwoven material may be, for example, a synthetic spunbonded nonwoven material. Alternatively, nonwoven material may be any other suitable type of nonwoven material, such as, for example, a spun-melted nonwoven material, a wet laid nonwoven material, a dry laid nonwoven material, a needle punched nonwoven material, or a melt blown nonwoven material. Nonwoven material may be constructed using any suitable fiber composition, such as, for example, nylon, polyester, polypropylene, rayon, cellolosic, polyamide, acrylic, polyethylene, cotton, wool, any other suitable fiber composition, or a combination of such fiber compositions. Nonwoven material may have a weight in the range of 0.25 to 1.0 ounces per square yard. In certain instances, the nonwoven material may have a weight in the range of 0.3 to 0.5 ounces per square yard, 0.25 to 0.6 ounces per square yard, 0.4 to 0.7 ounces per square yard, 0.6 to 1.0 ounces per square yard, or any other suitable range. An example of an illustrative nonwoven material that may be used in the backing layer of the laminated article is a spunbonded polypropylene nonwoven material obtained from First Quality Nonwovens, Inc. (Great Neck, N.Y.).

Elastic fabrics can also be used. For example, various elastic warp knit fabrics are known, wherein non-elastic yarn is formed into a fabric or a mesh to bind and hold laid-in elastic threads within the structure in a stretched state to impart elastic properties to the fabric structure. Other elastic warp knit fabrics are known, wherein the structure is formed from stitches which have non-elastic and elastic thread components. Each individual elastic thread is a component of only one stitch in a course. Fabrics with laid-in elastic yarn typically suffer from a high incidence of streaks if the non-elastic yarn is knit with tension on the non-elastic yarn low enough to produce a soft hand-feel in the fabric. Fabrics with laid-in elastic yarn can be engineered to have good stretch and modulus properties in the length of the fabric, but generally they have lower stretch properties in the width of the fabrics. Fabrics with single strands of elastic yarn formed into stitches with the non-elastic yarn generally have a high incidence of streaks because of the non-consistent response of the elastic yarn in the stitches. They can also be more costly because they require larger quantities of expensive elastic yarn for a given fabric weight. They generally have relatively long stretch properties, but a relatively high modulus. Warp knit elastic fabrics are also known, wherein a knitted ground construction composed of a plurality of pairs of non-elastic warp threads are formed into a plurality of wales and courses of single thread stitches one thread of each of the pairs forming stitches in adjacent wales and alternate courses, and wherein the other thread of each of the pairs forms stitches in non-adjacent wales and alternate courses. A plurality of elastic threads extending between the wales generally parallel thereto, are inlaid in the ground construction with a non-elastic warp thread of the ground construction wrapped about each of the elastic threads to maintain the elastic threads in the ground construction. There are also known other elastic warp knit fabrics, comprised of a plurality of courses of elastic and non-elastic threads in which each of the elastic threads is knitted into every stitch across the width of the fabric in consecutive courses. There are also known other elastic warp knit fabrics, wherein a ground construction composed of a single non-elastic yarn system is used to bind and conceal laid-in elastic yarns from a single yarn system in such a way as to reduce the danger of the non-elastic yarn in the knitted ground structure from raveling.

Elastic Layer

In embodiments having an elastic layer, which may be considered to be part of the backing or separate from the backing, the elastic layer may include a sheet, yarn, and/or strand material that is capable of sustaining deformation without a permanent, detrimental loss of size or shape. Materials suitable for use as the elastic layer include a wide variety, but not limited to, of elastic threads, yarn rubber, flat rubber (e.g. as bands), elastic tape, film-type rubber, polyurethane, and, tape-like elastomer, or foam polyurethane or formed elastic scrim. The elastic layer may be unitary, multipart, or composite in construction. Threads or ribbons, where used, may be multiple and may be applied as a composite. The elastomerics used in the elastics may be latent and nonlatent.

Alternatively, stretch yarns, such as elastic stretch yarns or thermoplastic stretch yarns, can be used along the length of the fabric, preferably in the wale, to impart extensibility. Elastic stretch yarns, such as Lycra, Spandex, polyurethanes, and natural rubber, could be used as described in U.S. Pat. No. 4,668,563 (Buese). Thermoplastic stretch yarns, such as polyesters and polyamides, could also be used as described in U.S. Pat. No. 4,940,047 (Richter et al.).

Referring to FIGS. 1 and 2, the elastic strands 16 may be a 210 denier spandex yarn, such as that sold by Creora, Hyosung Spandex Co., Ltd. (Korea). Another elastic yarn that may be used is a 280 denier elastic yarn that is sold under the trademark GLOSPAN (Globe Mfg. Co., Fall River, Mass.). Depending on the amount of elasticity desired in the finished article 10, both the denier and number of elastic strands per inch (measured transversely) of the tape/bandage may vary. For example, the denier of the elastic strands may vary from less than 100 to about 1000, and there may be from about 5 to about 15 elastic strands per inch. In some embodiments, the elastic strands may be characterized by the ability to stretch anywhere from 700 to 800% of their original unstretched length before they fail; may have a tensile strength of about 200 to 300 grams in a standard tensile strength test; and/or may contribute about 4 to 10 grams of weight per square meter of the overall article, for example, about 6.5 grams/m² of the article.

In embodiments including an elastic layer, the elastic layer may be positioned between the backing layer and the foam layer, and may be permeated with the cohesive composition. However the elastic layer can also be secured to the foam and/or backing layers with other compositions or techniques. The elastic layer need not be used in concert with a backing layer, and can be secured to either side of the foam layer, as discussed in greater detail above.

Foam Wound Care Pad

In some embodiments, the foam layer cohesive article includes a foam pad which is distinct from the foam layer detailed above. The foam pad can be applied directly to an open wound, skin ulcer, or sore on a particular body part, and used to absorb fluids emitted from such wound, ulcer, or sore. The foam pad is typically attached to a portion of the foam layer cohesive article, and in use the foam pad is placed over the wound, sore, or ulcer, and the remainder of the article is wrapped around the afflicted body part, for example twice or more. The article compresses the pad against the wound both securely and comfortably. The pad can also be used with other cohesive articles that do not necessarily include a foam layer, for example the CO-FLEX or POWERFLEX articles described above.

FIG. 4 is a top view of a foam layer cohesive article having foam wound care pad for direct application to an open wound, according to one embodiment of the invention. The foam wound care pad 20 is attached to the foam layer elastic cohesive bandage 30, for example attached with an adhesive agent, and can be applied directly to an open wound or ulcer and secured in place by winding the foam layer cohesive bandage securely around the affected area.

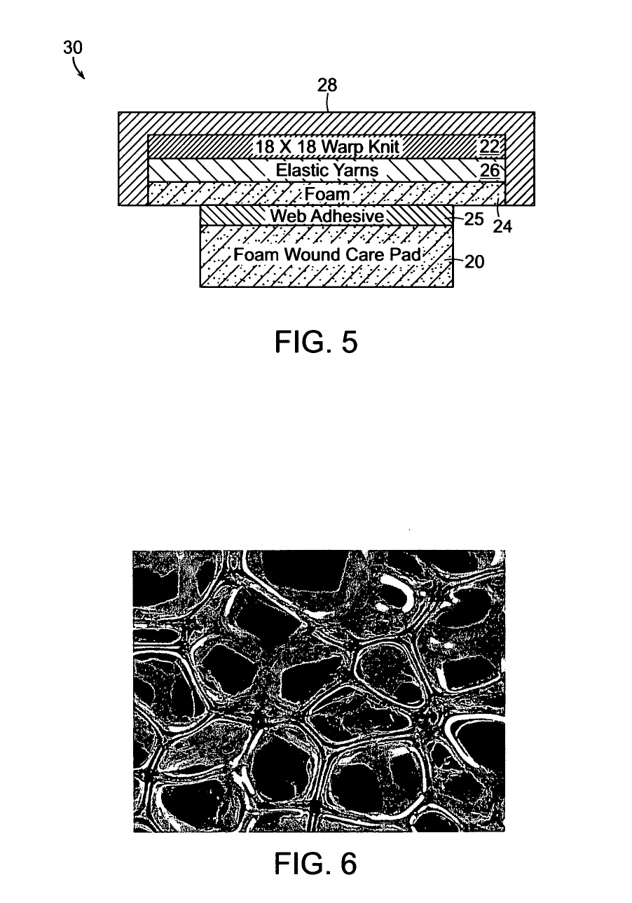

FIG. 5 is a cross-sectional view at line 3-3 of FIG. 4 showing the foam wound care pad 20 attached to the foam layer 24 of the foam layer cohesive bandage 30 with a standard web adhesive 25. The cohesive agent 28 permeates the 18×18 warp knit backing 22, the elastic yarn layer 26 (spandex yarns extending longitudinally), and the thin foam layer 24 of the foam layer cohesive bandage structure 30.

Depending on the application, the foam pad is preferably hydrophilic, and provides “wicking” of fluids, such as wound exudate, with which it is in contact. In particular, in many applications the foam pad has the ability to transport liquids such as wound exudate from the area of the wound itself through the foam, e.g., to the overlying surface of the foam layer cohesive bandage overwrap. The foam layer cohesive bandage overwrap would typically be adjacent to the surface of the pad that is opposite to the surface contacting the wound. The word “contact” as used in the present application will be used interchangeably with the term “fluid contact”, to mean that the pad is capable of wicking fluids from the wound site regardless of the presence of an interface material, such as a stockinette or gauze, between the pad and the wound. Foam wound care pads are compatible with many such interface materials, so long as the material that does not interfere with fluid contact between the wound site and the pad to the point where the foam pad would not serve its intended purpose.

Useful foam pads typically demonstrate significant, and preferably substantial, hydrophilicity, such as open cell polyurethane, polyethylene and silicone foam pads. of the foam pads may be pliant, extensible, and/or have an open-celled structure. As used herein, the term “open-celled” refers to a foraminous structure having interconnecting or communicating orifices or cavities therein caused by a sufficient number of the wall membranes of the foam cells having been removed. Further, as used herein, the word “impregnated” and inflected forms thereof refers to the condition in which an agent is intermingled with and in surrounding relation to the wall membranes of the cells and the interconnected cells of the blank.

The foam pad can comprise any one of a number of extensible foams that are open-celled, such as polyether- or polyester-based polyurethane foams. In applications where the foam pad is intended to absorb exudate from a wound, the porosity of the foam pad is selected in order to absorb a sufficient amount of wound exudate. For example, in some embodiments, the foam pad may have from about 10 to about 50 pores per centimeter (i.e., about 30 to about 120 pores per inch), or about 20 to 40 pores per centimeter. As used herein, the term “pores per centimeter” refers to the average number of pores located along a linear centimeter of the foam sheet. The number of pores per linear centimeter may be determined in a number of ways known to those skilled in the art, for example, by photomicrographic means, or by measuring the foam's resistance to air flow or a pressure differential, and using such information to calculate the approximate number of pores in the foam.

When the number of pores per centimeter is decreased below about 10, a foam may feelcoarse or rough, and may not hold enough wound exudate or provide the necessary strength for the resulting pad or to retain the desired conformation. It will be understood, however, that the desired number of pores per centimeter parameter is related to the ability of the foam pad to absorb exudate so as to provide sufficient properties for use as a wound dressing pad. In some applications, the pad may not be intended to absorb exudate, in which case the number of pores per centimeter of the pad, or even whether the pad is hydrophilic, is not a significant consideration in selecting the pad for that application. Instead, the pad may be selected on the basis of its comfort against the skin or its thickness, for example.

The dimensions of the foam pad depends in large part on the intended use of the pad. For example, a foam layer cohesive article having a foam wound care pad can be prepared and packaged having dimensions intended for use in apposition to a particular type and/or size of body part. One dimension will related to he thickness of the affected body part, i.e., the distance(s) between the major surface to be contacted with the body part, and the opposite surface thereto. The length of the overlying foam layer cohesive bandage can be adjusted accordingly. The dimensions of the foam pad required depend upon the surface area of the wound or ulcer to be supported and/or treated, and can be varied as desired, as apparent to those skilled in the art. The foam wound care pad can generally be trimmed, as with a blade or scissors, or by grinding or abrading, or even by hand tear, to provide a desired size and shape. For example, an article intended to be applied to a finger might have a length of, e.g., 5-10″, suitable for multiple wraps around an average finger, and a pad of a length of, e.g., 1-2″, suitable for less than one wrap around an average finger. Or, for example, an article intended to be wrapped around a torso might have a length of, e.g., 2-5 yards. These values are intended to be exemplary. In many embodiments, the article will have a length allowing at least two and possibly several wraps around a desired body part of average size (e.g., an average arm, leg, finger, or torso), and a foam pad that extends less than one wrap around the desired body part, although any desired sizes of the article or pad are possible. The article may have any desired width. Commercially available articles generally come in 1″, 1.5″, 2″, 3″, 4″, and 6″ widths, although other sizes are possible.

In some embodiments, the foam pad will have a thickness between about 5/16″ (˜0.8 cm), and can range from about 0.4 cm to about 5 cm, e.g., between about 0.6 cm and about 2 cm. The foam sheet need not be of uniform thickness, particularly in situations, for example, where a portion of a body part requires additional support or cushioning. The pad is, desirably, sufficiently dimensioned to encompass the area of the body part to be covered.

The foam pad utilized may have a density in the range of about 0.02 to about 0.15 g/cm³, and most usefully, between about 0.02 and about 0.07 g/cm³. Examples of suitable foam pads include “E-100”, “E-290”, “P-60”, “P-80” and “P-100”, each available from Illbruck U.S.A., Minneapolis, Minn. Another material that can be used for the foam pad of the present invention is a polyether-based polyurethane foam sheet that is approximately 2 cm thick and is presently available from Illbruck USA, as type “E-150”.

These hydrophilic foam compositions may be prepared by any means known in the art, such as by foaming prepolymers by means of the addition of chemical or physical blowing agents. Accordingly, hydrophilic polyurethane compositions may be prepared either by foaming isocyanate-capped prepolymers by the addition of water, or by frothing aqueous dispersions of fully reacted polyurethane polymers to entrap chemically inert gases therein. These foam compositions must be prepared, of course, with the understanding that any types or amounts of additives, introduced to confer or improve hydrophilicity or other characteristics of the foam, will not result in medically unacceptable cytoxicity in the ultimate composition so produced. For example, the following surfactants may be used to enhance hydrophilicity in the preparation of hydrophilic foam compositions for use in the present invention: sorbitan trioleate; polyoxyethylene sorbitan oleate; polyoxyethylene sorbitan monolaureate, polyoxyethylene lauryl ether; polyoxyethylene stearyl ether; fluorochemical surfactants such as Zonyl FSN by E. I. du Pont and Fluorad FC 170C by 3M, and block copolymer condensates of ethylene oxide and propylene oxide with propylene glycol, such as the PLURONIC surfactants available from BASF Wyandotte.

In addition, the foam pad compositions may be thermoplastic, and thus reversibly soften upon heating. In some embodiments, the compositions will soften and become tacky, or at least self-adherent, between 225° F. and 300° F., although compositions may be used which soften between 200° F. and 350° F., and at the same time demonstrate thermal stability at ordinary room temperatures.

Further, the foam compositions may be cast or skived into low-density sheets. In particular, sheets formed from these compositions may have a density between 4 and 20 lbs/ft³, more usefully between 5 and 12 lbs/ft³, e.g., 8 lbs/ft³. The low density of the foam pad contributes both to the lightweight absorbency of the foam bandage and the low cost of the materials necessary in the manufacture thereof. As discussed above, the low density foams may be open-celled or partially open-celled, as long as the foams are liquid permeable in contrast to the rigid impermeable closed-cell foams, however the desired level of permeability will depend on the desired application.

Some useful foam pads include polyurethanes, including those which result from foaming isocyanate-capped prepolymers and those prepared by frothing aqueous polyurethane dispersions. Foam pads prepared by mechanically frothing, casting and curing aqueous polyurethane dispersions are also useful, e.g., foam pads recognized in the art as ionically water dispersible are particularly useful.

One useful system for preparing aqueous ionic polyurethane dispersions is to prepare polymers that have free acid groups, preferably carboxylic acid groups, covalently bonded to the polymer backbone. Neutralization of these carboxyl groups with an amine, preferably a water soluble monoamine, affords water dilutability. Careful selection of the compound bearing the carboxylic group must be made because isocyanates, the reactive group employed most often in the generation of urethane linkages, are generally reactive with carboxylic groups. However, as disclosed in U.S. Pat. No. 3,412,054, incorporated herein by reference, 2,2-hydroxymethyl-substituted carboxylic acids can be reacted with organic polyisocyanates without significant reaction between the acid and isocyanate groups as a result of the steric hindrance of the carboxyl by the adjacent alkyl groups. This approach provides the desired carboxyl-containing polymer with the carboxylic groups being neutralized with the tertiary mono-amine to provide an internal quaternary ammonium salt and, hence, water dilutability.

Suitable carboxylic acids and, preferably, the sterically hindered carboxylic acids, are well-known and readily available. For example, they may be prepared from an aldehyde that contains at least two hydrogens in the alpha position which are reacted in the presence of a base with two equivalents of formaldehyde to form a 2,2-hydroxymethyl aldehyde. The aldehyde is then oxidized to the acid by procedures known to those skilled in the art.

The polymers with the pendant carboxyl groups are characterized as anionic polyurethane polymers. However, an alternate route to confer water dilutability is to use a cationic polyurethane having pendant amino group. Such cationic polyurethanes are disclosed in, for example, U.S. Pat. No. 4,066,591, incorporated herein by reference.

Useful polyurethanes can be made, e.g., by reacting di- or polyisocyanates and compounds with multiple reactive hydrogens suitable for the preparation of polyurethanes. Such diisocyanates and reactive hydrogen compounds are more fully disclosed in U.S. Pat. Nos. 3,412,054 and 4,046,729, the entire contents of which are incorporated herein by reference. Further, the processes to prepare such polyurethanes are well recognized as exemplified by the aforementioned patents. Aromatic, aliphatic and cyclo-aliphatic diisocyanates or mixtures thereof can be used in forming the polymer. Such diisocyanates, for example, for tolylene-2,4-diisocyanate; tolylene-2,6-diisocyanate; meta-phenylene diisocyanate; biphenylene-4,4′-diisocyanate; methylene-bis-(4-phenol isocyanate); 4,4-chloro-1,3-phenylene diisocyanate; naphthylene-1,5-diisocyanate; tetramethylene-1,4-diisocyanate; hexamethylene-1,6-diisocyanate; decamethylene-1,10-diisocyanate; cyclohexylene-1,4-diisocyanate; isophorone diisocyanate and the like. Arylene and cycloaliphatic diisocyanates are particularly useful.

In some embodiments, the polyurethane foam can be produced using a dispersion viscosity that is generally in the range of from 10 to 1000 centipoise. Useful solutions of polyurethane in organic solvents, by contrast, generally have viscosities of several thousand centipoise, ranging as high as 50,000 centipoise when the solution contains about 20 to 30 percent by weight polyurethane. Useful polyurethane dispersions contain, moreover, about 50 to 75 percent by weight polyurethane solids in dispersion. A particularly useful polyurethane concentration is 55 to 70 percent by weight and the most preferred concentration is 65 percent by weight polyurethane solids in dispersion.

Particularly useful polyurethane dispersions include the non-crosslinked polyurethane compositions recited in U.S. Pat. No. 4,171,391, incorporated herein by reference. Other useful polyurethane dispersions include those available from Witco Chemical Company under the trade designation Witcobond® W-290H; these dispersions yield foams which demonstrate inherent hydrophilicity, even in the absence of surfactants. The Witcobond® W-290H dispersions contain 65 percent by weight anionic polyurethane solids having particulate diameters less than 5 microns.

Use of Article with Unna Boot Medicated Pad

Foam layer cohesive articles can also be used to secure a medicated “Unna boot” pad to a body part. An Unna boot is a moist, gauze bandage carrying calamine lotion and, optionally, zinc oxide and/or glycerine. The original Unna boot was first described in 1854 and named for its inventor. The Unna boot medicated pad promotes healing of ulcers, such as venous ulcers by reducing infection and increasing the return of blood to the heart. For venous leg ulcers, the Unna boot is wrapped from the toes to just below the knee, covering the ulcer and the lower leg. The gauze then dries and hardens.

Conventional latex-free cohesive articles art are generally incompatible with Unna boots, as is well known in the art. If a conventional latex-free cohesive article is attempted to be used to secure an Unna boot to a body part, the article rapidly loses its cohesive properties and subsequently unravels. Specifically, as the calamine lotion seeps through the article, it breaks cohesive bonds between overlying layers of the wrapped article. In contrast, a foam layer cohesive article—even one with a latex-free cohesive composition—can be successfully used with an Unna boot. The foam layer cohesive article satisfactorily retains its cohesion when used to wrap an Unna boot to a body part.

Without wishing to be bound by theory, it is believed that one factor contributing to the successful use of the foam layer cohesive article with an Unna boot is that the foam layer in the article slows the speed of the lotion in the Unna boot from seeping through the article as it is being wrapped around the body part. This may allow the cohesive composition in the article to form a cohesive-to-cohesive contact between underlying/overlying layers of the article, and then the tiny “suction cups” in the open cell structure of the foam form a secure cohesive bond to underlying/overlying layers, and the lotion cannot penetrate these bonds.

Thus, the foam layer cohesive article may be wrapped snugly over the Unna boot. A foam layer cohesive bandage/Unna boot dressing is applied every one to two weeks until the ulcer is healed. Initially, more frequent changes may be required for heavily draining ulcers.

An exemplary Unna boot pad is the GELOCAST™ Unna's Boot Dressing which is a non-raveling gauze preparation carrying a soothing zinc oxide/calamine formulation that provides firm compression therapy promoting the healing of irritated or ulcerated skin (BSN-Jobst Gelocast Unna Boot Dressing—4″×10 yds. available from the Medical Supply Company (Alpharetta, Ga.).

Other Unna boot preparations include the Unna's Boot commercially available from Biersdorf, Inc., which comprises a zinc paste-containing bandage wrapped around a patient's leg from above the toes to below the knee. Still other Unna's Boot/zinc impregnated treatments are available from Miles and Graham Field. These dressings are often left in place for a week at a time and typically require the use of absorbent pads that must be applied to the outside of the dressings in the area of the ulcer to absorb excess exudate. Seepage of exudate throughout the wrap is common, and damage to the skin and epithelium may occur. The foam layer cohesive bandage of the invention is capable of absorbing this fluid, thereby providing therapeutic pressure to the wound while obviating the use of additional absorbent dressings.

Optional Sterilization of Foam Layer Cohesive Articles

Foam layer cohesive articles can optionally be sterilized using EtO (ethylene oxide) techniques known in the art, without detrimentally affecting the properties of the article. Typically, the well-known EtO process includes four basic phases. The four phases are: (1) air removal (vacuum), (2) steam injection and conditioning dwell, (3) EtO injection and gas dwell, and (4) gas purge and air inbleed. In the case of a conventional, rolled cohesive bandage, the multiple pressure and vacuum operations involved in the EtO process typically cause the bandage roll to shrink in size and compress, and can greatly increase the cohesive bond between overlying and underlying layers. If the bandage peel values become too high as a result of the sterilization process, the bandage will be extremely difficult if not impossible to remove from the roll. Thus, in order to limit the effects of this “squeezing” during the vacuum portion of the EtO process, conventional cohesive bandages are generally manufactured using lower peel values and rolled looser than manufacturers normally would for a bandage that would not be subjected to the EtO process.

In contrast, foam layer cohesive articles do not have this limitation. Without wishing to be limited by theory, it is believed that the closed cells that are inherently present in a foam (even an open-cell foam, as described above and as is known in the art) expand during the vacuum portion of the EtO process, and that this expansion keeps the individual layers of the foam separated when in roll form. This expansion may also keeps the open cells separated, which may otherwise have caused compression of the bandage. This feature, among other possible features, allows the bandage made with a foam layer to be manufactured using normal peel values and wound to normal tension levels.

After sterilization, the article can be packaged so as to maintain its sterility until use, using techniques that are known in the art. The article can also be packaged without requiring a sterilization step, for example using a flow wrap for a non-sterile product, or a Dupont Tyvek® 1059B for a sterile product.

### EXAMPLES

Various embodiments of the invention are further illustrated by the following examples, which should not be construed as limiting. In these illustrative examples, the construction of an exemplary foam layer bandage is described and its unique cohesive and adhesive properties are tested. In addition, the construction of an exemplary foam layer bandage with a hydrophilic foam wound care pad is described in detail.

5.1 Construction of Foam Layer Cohesive Bandage

A first exemplary foam layer cohesive bandage was constructed as shown in FIG. 3 and described in further detail below. A thin layer of commercial polyurethane foam 0.025 inches thick (product # 157320 (0.025″ thick×60″ wide) from W.T. Burnette & Co., Jessup, Md.) was dip coated in latex-free cohesive (43.8% solids, 676 cPs (centipoise, metric dynamic viscosity equal to 1 millispascal second (mPa·s)). The excess latex-free cohesive coating was removed by nip-processing to provide a final coating weight in the range of approximately 80 to approximately 100 g/m in the finished bandage. A first layer of warp knit Milliken open weave fabric (18 yarns per inch of 30 denier warp×18 yarns per inch of 70 denier weft) was attached to the cohesive-saturated foam layer. The foam layer surface of the resulting composite was then laminated to a layer of spandex elastic yarns (210 denier/569 from Hyosung Spandex Co., Korea) stretched to 120% of its relaxed length and a second layer of warp knit Milliken open weave fabric (18 yarns per inch of 30 denier warp×18 yarns per inch of 70 denier weft) and heat-treated as shown in FIG. 3.

The resulting flexible foam cohesive bandage includes about 14.4 g/m² of the first layer of warp knit Milliken open weave fabric as the first layer; about 22.3 g/m² of the thin layer of commercial polyurethane foam as the second layer; about 5.0 g/m² of the elastic yarns as the third layer; and about 14.4 g/m² of the second layer of warp knit Milliken open weave fabric as the fourth and final layer. Together with the coating weight of latex-free cohesive of about 35.0 g/m², the resulting composite foam layer cohesive bandage was approximately 91 g/m².

One exemplary foam layer cohesive article was constructed as shown in FIG. 3 and described in further detail below. A first layer of warp knit Milliken open weave fabric (18 yarns per inch of 30 denier warp×18 yarns per inch of 70 denier weft) was dip coated in a tan non-latex cohesive (43.% solids, 1000 cPs (centipoise, metric dynamic viscosity equal to 1 millispascal second (mPa·s)). A second layer of spandex elastic yarns (210 denier/569 from Hyosung Spandex Co., Korea) stretched to 120% of its relaxed length was then mated to the warp knit layer. A third thin layer of commercial polyurethane foam 0.025 inches thick (product # S82F (0.025″thick×60″ wide) from W.T. Burnette & Co., Jessup, Md.) was saturated with the cohesive along with the warp knit and spandex layer. The excess non-latex cohesive coating was removed by nip-processing to provide a final coating weight of non-latex cohesive in the range of approximately 80 to approximately 110 g/m² in the finished bandage and heat-treated as shown in FIG. 3.

The resulting flexible foam cohesive bandage included about 14.4 g/m² of warp knit Milliken open weave fabric as the first layer; about 5.0 g/m² of the elastic yarns as the second layer; and about 22.3 g/m² of the thin layer of commercial polyurethane foam for the third and final layer. Together with the coating weight of non-latex cohesive of about 65.3 g/m², the resulting composite foam layer cohesive bandage was approximately 107 g/m².

Another exemplary foam layer cohesive article was constructed as shown in FIG. 3 and described in further detail below. A first layer of non woven (10 GSM) was dip coated in a white latex cohesive (40.% solids, 650 cPs (centipoise, metric dynamic viscosity equal to 1 millispascal second (mPa·s)). A second layer of spandex elastic yarns (210 denier/569 from Hyosung Spandex Co., Korea) stretched to 120% of its relaxed length and a third layer of a thin commercial polyurethane foam 0.025 inches thick (product # SW282JJ (0.025″ thick×60″ wide) from W.T. Burnette & Co., Jessup, Md.) was saturated with the cohesive along with the non-woven and spandex layer. The excess latex cohesive coating was removed by nip-processing to provide a final coating weight of latex cohesive in the range of approximately 60 to approximately 80 g/m² in the finished bandage and heat-treated as shown in FIG. 3.

The resulting flexible foam cohesive bandage included about 10 g/m² of non-woven fabric as the first layer; about 5.0 g/m² of the elastic yarns as the second layer; about 22.3 g/m² of the thin layer of commercial polyurethane foam as the third and final layer. Together with the coating weight of latex cohesive of about 34.7 g/m², the resulting composite foam layer cohesive bandage was approximately 72 g/m².

Another exemplary foam layer cohesive article was constructed as shown in FIG. 3 and described in further detail below. A first layer of warp knit Milliken open weave fabric (18 yarns per inch of 50 denier warp×13 yarns per inch of 150 denier weft) was dip coated in a white latex cohesive (40.% solids, 750 cPs (centipoise, metric dynamic viscosity equal to 1 millispascal second (mPa·s)). A second layer of spandex elastic yarns (210 denier/569 from Hyosung Spandex Co., Korea) stretched to 120% of its relaxed length was then mated to the warp knit layer. A third thin layer of commercial polyurethane foam 0.032 inches thick (product # SW282JJ (0.032″thick×60″ wide) from W.T. Burnette & Co., Jessup, Md.) was saturated with the cohesive along with the warp-knit and spandex layer. The excess latex cohesive coating was removed by nip-processing to provide a final coating weight of latex cohesive in the range of approximately 80 to approximately 100 g/m² in the finished bandage and heat-treated as shown in FIG. 3.

The resulting flexible foam cohesive bandage included about 14.4 g/m² of warp knit Milliken open weave fabric as the first layer; about 5.0 g/m² of the elastic yarns as the second layer; and about 22.3 g/m² of the thin layer of commercial polyurethane foam the third and final layer. Together with the coating weight of latex cohesive of about 49.3 g/m², the resulting composite foam layer cohesive bandage was approximately 91 g/m².

Another exemplary foam layer cohesive article was constructed as shown in FIG. 3 and described in further detail below. A first layer of warp knit Milliken open weave fabric (18 yarns per inch of 30 denier warp×16 yarns per inch of 70 denier weft) was dip coated in a tan non-latex cohesive (43.% solids, 1000 cPs (centipoise, metric dynamic viscosity equal to 1 millispascal second (mPa·s)). A second layer of spandex elastic yarns (210 denier/569 from Hyosung Spandex Co., Korea) stretched to 120% of its relaxed length was then mated to the warp knit layer. A third thin layer of commercial polyurethane foam 0.025 inches thick (product # SW282JJ (0.025″ thick×60″ wide) from W.T. Burnette & Co., Jessup, Md.) was saturated with the cohesive along with the warp-knit and spandex layer. The excess non-latex cohesive coating was removed by nip-processing to provide a final coating weight of non-latex cohesive in the range of approximately 80 to approximately 110 g/m² in the finished bandage and heat-treated as shown in FIG. 3.

The resulting flexible foam cohesive bandage included about 14.4 g/m² of warp knit Milliken open weave fabric as the first layer; about 5.0 g/m² of the elastic yarns as the second layer; and about 22.3 g/m² of the thin layer of commercial polyurethane foam the third and final layer. Together with the coating weight of non-latex cohesive of about 56.3 g/m², the resulting composite foam layer cohesive bandage was approximately 98 g/m².

5.2 Properties of Foam Layer Cohesive Bandage

The first exemplary foam layer bandage was tested for its cohesive properties (self-stickiness) using cohesive bond tests adapted from standard peel force bond and shear force bond tests as described by the ASTM (West Conshohocken, Pa.). In addition, the tensile strength and percentage stretch of the bandage were tested using standard methods known in the art.

For example, under the well-known conventional ASTM methods, the cohesive bond strength of a finished elastomeric product can be determined by a T-Peel test. In such a test, two strips of the finished elastomeric product measuring 1 inch in width and of equal length, are placed face to face and a cylindrical weight rolled across the surface of the superimposed strips. The two non-superimposed ends are clamped in the jaws of a tensile testing apparatus and pulled linearly in opposite directions, pulling the two strips apart. The resistance of the superimposed strips to the movement of the clamps is measured in ounces/inch of width. The ASTM D-3330 and PSTC-1 tests are written for adhesive tape, as compared to cohesive bandages, but can be modified by substituting the stainless steel for the back side of the bandage, thus providing front to back peel values for a layered cohesive article. The cohesive bond strength was approximately 9.55 oz/in-w peel force and approximately 8.64 lb/2 in², providing a secure fit when wound upon itself with very minimal adhesion to skin. In addition, the tensile strength was approximately 15.24 lb/in-w) and a percentage stretch of approximately 117%.

The foam layer bandage was also tested for its ability to stick to nonporous surfaces such as glass, steel and plastic. While conventional cohesive tapes and bandages do not provide any significant grip to such heterologous surfaces, the foam layer bandage of the invention was surprisingly capable of lightly bonding with such smooth surfaces as measured with a standard stainless steel surface using testing protocols known in the art, here ASTM D-3654. An exemplary foam layer cohesive bandage, as constructed in Example 5.1, was tested for its cohesive properties, as well as its ability to adhere to a stainless steel surface, using peel force bond strength and shear force bond strength tests. As expected, the “cohesive” peel force bond strength of the tape wound upon itself front to back was approximately 16.2 oz/in-w following compression of the two layers using four passes of a 10 lb roller. Also as expected, the “cohesive” shear force bond strength of the tape wound upon itself front to back was approximately 13.75 lb/2-in² following compression of the two layers using four passes of a 4.5 lb roller.

Unexpectedly, however, the “adhesive” peel force bond strength of the tape to a stainless steel surface was approximately 7 oz/in-w following compression of the two layers using four passes of a 4.5 lb roller, and approximately 1.1 oz/in-w following compression of the two layers using four passes of a 10 lb roller. While relatively weak, this measurable light adhesion to smooth surfaces provides a unique property in a cohesive bandage. In particular, this property will provide grip to braces, splints and other types of support devises that are wrapped with the foam layer cohesive elastic bandage. Conventional cohesive bandages do not provide such grip, and thus do not provide as secure a dressing around such support devices as do the foam layer cohesive bandages of the invention.

While not wishing to be bound by a single theory of operability, the unusual light adhesive properties of the foam layer cohesive bandage to nonporous smooth surfaces may arise from the coating of inside surfaces of exposed open cells of the foam with the cohesive agent occurring during the manufacturing process. In particular, the cohesive-coated open foam cells appear to create miniature “suction cups” that allow for light adhesion to the nonporous surface.

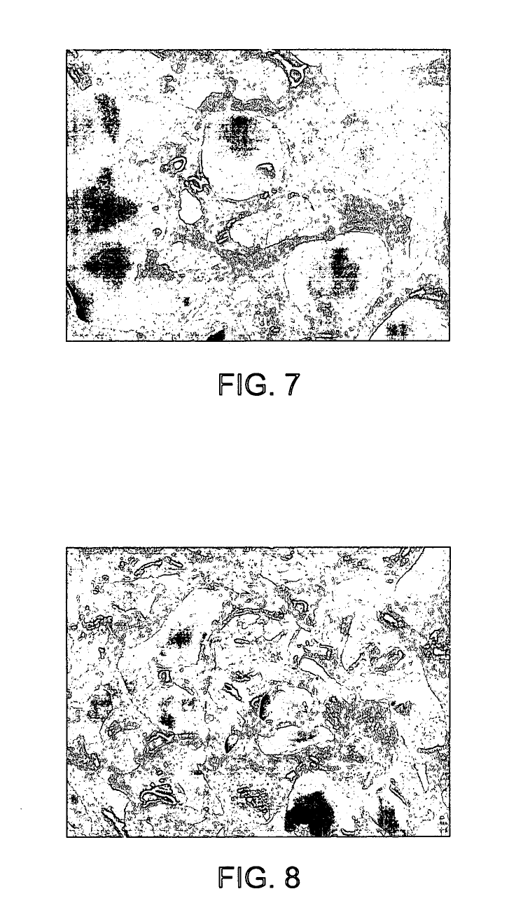

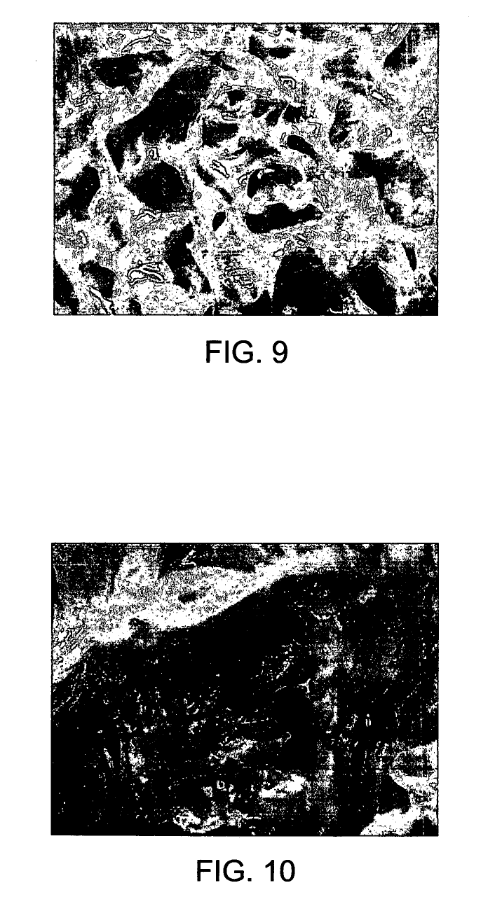

FIG. 6 is a microscope image of an uncoated foam layer having a thickness of about 0.025 inches. It clearly shows both the open and closed cells of the foam. FIG. 7 is a microscope image of the foam layer side of an article having a foam layer with thickness of about 0.025 inches, an 18×18 warp-knit (weft-insertion) fabric, elastic yarns, and a latex-free cohesive composition according to one embodiment of the invention. The bandage is in a relaxed unstretched state and this image shows the coated open and closed cells of the foam. FIG. 8 is a microscope image of the foam layer side of the article of FIG. 7, the foam layer being compressed onto a glass slide (image taken through the glass slide). This shows the compressed foam cells that are providing the suction that is holding the cohesive bandage to the slide. FIG. 9 is a microscope image of the foam layer side of the article of FIG. 7, the foam layer side being compressed onto a glass slide (image taken through the glass slide). FIG. 10 is a microscope image of the warp-knit (weft-insertion) fabric side of the article this is showing that there is still exposed foam layer thru the warp knit which provides a foam to foam bond which accounts to the excellent quick stick ability of the foam of FIG. 7, the foam layer side being compressed onto a glass slide.

FIGS. 6-10 were taken with a compound microscope with a 4× objective using a Paxcam attached camera. The warp knit in the picture is approximately 0.010 inches edge to edge in the picture (it tends to flatten a little in processing) The foam cells are also approximately 0.010 inches edge to edge (on avg). All the pictures were taken using the same objective.

5.3 Construction of Foam Layer Cohesive Bandage with a Foam Wound Care Pad

A foam layer cohesive bandage having a foam wound care pad for direct application to an open wound was constructed as described in further detail below. The structure of the finished foam layer cohesive bandage with foam wound care pad is shown in FIGS. 4 and 5.

Briefly, the foam wound care pad is constructed using a commercially-available hydrophilic foam pad (W.T. Burnette and Co., Jessup Md., 5/16 inch thick and having a density of about 8 lbs/foot³). Open cell polyethylene or open cell silicone pads may also be used. The web adhesive used to attach the hydrophilic foam pad to the foam layer cohesive bandage is a commercially-available urethane adhesive film from Adhesive Films, Inc. (Pine brook, NJ) UAF-425 .003″ in finished thickness. The foam in combination with the web adhesive used provides an almost water proof barrier between the foam and the out side of the bandage. The foam wound care pad absorbs wound exudate and retains the ability to pucker as it absorbs fluid, which provides additional therapeutic pressure to the wound. The bandage and the web adhesive do not prevent the foam pad from absorbing exudate fluids.

To process large quantities of the foam layer cohesive bandage having a foam wound care pad (LF³ foam bandage), the foam pad is added during a rewind process to a full rewind log of 54 inches. The web adhesive is attached to the foam pad, and then the foam pad is placed on the rewinder with a heat press and wound onto the foam layer cohesive bandage, which is then cut to size. The foam pad can be placed anywhere on the bandage roll and on any size bandage roll. The foam pad can be any size.

The foam pad can be also be attached with cohesive on one side and then allowed to dry and heat-pressed onto the bandage for a permanent bond. This version may not be as water resistive as the version in which a web adhesive is used to attach the foam pad to the foam layer cohesive bandage, but may exhibit enhanced stretch characteristics.

Another method of attachment is to coat the foam pad with a cohesive agent such as that used in the construction of the foam layer cohesive bandage. After drying, the resulting foam pad is cohesively, and reversibly, attached to the foam layer cohesive bandage and can readily be repositioned to suit the needs of the dressing application.

This version of the foam pad dressing has a sufficiently thin wound care pad to allow the foam pad to be hand torn. The bandage can also be made in to different colors by coloring the cohesive and or the foam. The bandage can be printed on to provide bandages having various novel and amusing designs.

The foam pad can be used for wound care or as a protective pad to prevent injury or ulceration. Different types of foam can be used in different applications. For example, open cell foams can be used for wound treatment, while closed cell foams can be used for protection.

The patent and scientific literature referred to herein establishes knowledge that is available to those of skill in the art. The issued U.S. patents, allowed applications, published foreign applications, and references, which are cited herein, are hereby incorporated by reference to the same extent as if each was specifically and individually indicated to be incorporated by reference.

Those skilled in the art will recognize, or be able to ascertain, using no more than routine experimentation, numerous equivalents to the specific embodiments described specifically herein. Such equivalents are within in the scope of the following claims.

What is claimed is:

1. A medical bandage comprising:
an elastically extensible article having first and second oppositely-facing exterior surfaces each of which is coated with a cohesive composition; and
an absorbent pad juxtaposed with and secured to one of the exterior surfaces of the extensible article.

2. The medical bandage of claim 1, the absorbent pad having a thickness not less than three times the thickness of the elastically extensible article and covering less than one-third of the surface of the article to which it is secured.

3. The medical bandage of claim 2, wherein the absorbent pad has a thickness greater than 0.1 inches and is sized and adapted to cover a wound on a body part.

4. The medical bandage of claim 3, wherein the absorbent pad has a length less than that of the circumference of the body part.

5. The medical bandage of claim 4, wherein the article has a length at least twice the circumference of the body part.

6. The medical bandage of claim 1, wherein the absorbent pad comprises hydrophilic foam.

7. The medical bandage of claim 6, wherein the hydrophilic foam comprises a plurality of open cells.

8. The medical bandage of claim 1, wherein the absorbent pad comprises at least one of polyurethane, silicone, polyethylene, and gauze.

9. The medical bandage of claim 1, wherein at least one of an adhesive, a cohesive, and a web adhesive secures the pad to the surface.

10. A package having the medical bandage of claim 1 sealed therein.

11. The package according to claim 10, wherein the medical bandage is in a sterile condition.

12. The medical bandage of claim 1 wherein the article is wound into a roll such that the first surface is cohesively attached to the second surface.

13. A package having the medical bandage of claim 12 sealed therein, the bandage being in a sterile condition.

14. The medical bandage of claim 1 having only one absorbent pad.
